(19) United States
(12) Patent Application Publication
(10) Pub. No.: US 2023/0096656 A1
(43) Pub. Date: Mar. 30, 2023

(54) RESISTIVE COATING DEVICE AND METHOD

(71) Applicants: Kester Julian Batchelor, Mound, MN (US); Teo Heng Jimmy Yang, Heath (GB); Bryan Tissington, Cardiff (GB)

(72) Inventors: Kester Julian Batchelor, Mound, MN (US); Teo Heng Jimmy Yang, Heath (GB); Bryan Tissington, Cardiff (GB)

(21) Appl. No.: 17/932,770

(22) Filed: Sep. 16, 2022

Related U.S. Application Data
(60) Provisional application No. 63/261,790, filed on Sep. 29, 2021.

(51) Int. Cl. A61B 18/14; A61B 18/12; C09D 183/04; C09D 5/24; C09D 7/61

(52) U.S. Cl. CPC A61B 18/1445; A61B 18/1206; C09D 183/04; C09D 5/24; C09D 7/61; A61B 2018/00571

(57) ABSTRACT

Electrosurgical devices are shown with a coated electrode. Electrosurgical devices and methods of use are shown to provide a higher concentration of energy at different resistance regions within a coating. Electrosurgical devices and methods of use are also shown to utilize heat in an electrode contained by a thermally insulative coating to provide a second tissue modification.

### PRIORITY CLAIM

This application claims the benefit of priority to U.S. Provisional Patent Application Ser. No. 63/261,790 Sep. 29, 2021, the contents of which are incorporated by reference in their entirety.

### BACKGROUND

Electrosurgery is the application of an electrical signal—an electrotherapeutic signal—to produce a change in biological tissue of a surgical patient in some manner. Various electrosurgical techniques are used to cut, coagulate, desiccate, or fulgurate the biological tissue. These electrosurgical techniques and others can be performed during various medical procedures, such as, for example, laparoscopic surgeries. These medical procedures include: appendectomy, cholecystectomy, colectomy, cystectomy, gastric banding, gastric bypass, hernia repair, nephrectomy, Nissen fundoplication, prostatectomy, sleeve gastrectomy, and others. Each of these medical procedures can have one or more electrotherapeutic phases, such as, for example, interrogation phase, heating phase, drying phase, cauterizing phase, etc.

The electrotherapeutic signals used in such medical procedures can be generated by an electrosurgical generator and then provided to the biological tissue via an electrosurgical instrument, which can be electrically connected to the electrosurgical generator. The electrosurgical instrument can be configured to mechanically and electrically engage the biological tissue to which the electrotherapeutic signal is provided. Various types of such electrosurgical instruments can be employed, including, for example, various types of forceps, conductive spatulas, electrical pads, etc.

Different medical procedures can implement different electrotherapeutic signals so as to achieve results specific to these different medical procedures. Various electrical metrics of the electrotherapeutic signals provided to the engaged biological tissue can be used to characterize these electrotherapeutic signals. These electrical metrics include: polarity (monopolar, bipolar), AC and/or DC, frequency, signal amplitude, attack and decay profiles, etc. Electrosurgical generators that generate these various electrotherapeutic signals can control one or more of these electrical metrics so as to provide electrotherapeutic signals that yield efficacious results in the biological tissue engaged by the electrosurgical instrument.

Components of electrosurgical devices, such as electrodes and other intended tissue contacting components, can be coated.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an electrosurgical system providing electrotherapy to biological tissue of a surgical patient.

FIG. 2 is a block diagram of an electrosurgical system for sealing biological tissue engaged by an electrosurgical instrument.

FIGS. 3A-3B are flow charts of a method for sealing a biological tissue engaged by an electrosurgical instrument.

FIG. 4A is a graph depicting examples of an electrical-power schedule used to control electrical power provided to biological tissue being sealed.

FIG. 4B is another graph depicting examples of an electrical-power schedule used to control electrical power provided to biological tissue being sealed.

FIG. 4C is another graph depicting examples of an electrical-power schedule used to control electrical power provided to biological tissue being sealed.

FIG. 5A shows selected portions of an electrosurgical device in accordance with some example embodiments.

FIG. 5B shows an example coating from FIG. 5A in accordance with some example embodiments.

FIG. 5C shows another example coating from FIG. 5A in accordance with some example embodiments.

FIG. 6 shows a surface including a hydrophobic physical structure in accordance with some example embodiments.

FIG. 7 shows another surface including a hydrophobic physical structure in accordance with some example embodiments.

FIG. 8 shows another surface including a hydrophobic physical structure in accordance with some example embodiments.

FIG. 9 shows examples of hydrophobic surface interactions in accordance with some example embodiments.

FIG. 10 shows a flow diagram of an example method in accordance with some example embodiments.

### DESCRIPTION OF EMBODIMENTS

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

Electrosurgically sealing or coagulating biological tissue engaged by an electrosurgical instrument is an electrosurgical technique used in various medical procedures. The engaged biological tissue can be electrosurgically sealed by heating the engaged biological tissue in a controlled manner. In some medical procedures, the biological tissue that is being sealed is a vessel. Heating of the vessel causes the collagen found in the vessel walls to become denatured. This denatured collagen forms a gel-like substance acting as glue between the vessel walls. When forced together and maintained together while cooling, opposite walls of a vessel will then form a seal.

Heating of the vessel is carefully controlled so that neither too little nor too much energy is provided to the vessel. If too much energy is provided thereto, then charring and/or burning of the vessel wall can occur. If too little energy is provided thereto, then seal quality of the vessel can be poor. One measure of seal quality is a pressure difference that the sealed vessel can withstand without bursting. Low quality seals can be compromised when the pressure applied thereto exceeds some value.

The rate at which the energy is provided to the vessel can also be carefully controlled so as to facilitate rapid performance of the electrosurgical procedure. Rapid performance of electrosurgical procedures reduces the time and difficulty of these procedures. The rate of heating, however, should not be so rapid as to cause uncontrolled boiling of fluid within the biological tissue. Uncontrolled boiling can rupture engaged or nearby biological tissues and/or compromise the quality of the seal.

Heating of the engaged biological tissue can be controlled by controlling the electrical power of an electrotherapeutic signal provided to and dissipated by the engaged biological tissue. Such electrical power can be controlled according to a sealing schedule. For example, the sealing schedule can be indicative of a product of a voltage difference across and an electrical current conducted by the engaged biological tissue. Thus, the sealing schedule is an electrical-power schedule. In some examples, the electrotherapeutic signal can be reduced or terminated in response to a termination criterion being met. In some examples, the termination criterion is a current characteristic, such as, for example, a decrease in current conducted by the engaged biological tissue. In some examples, the termination criterion is a resistance characteristic, such as, for example, an increase in the electrical resistance of the engaged biological tissue. Such an increase in the electrical resistance in excess of a predetermined delta resistance value can be used as a termination criterion, for example, where the predetermined delta resistance value is the difference between the measured resistance (or impedance) and the lowest value of the resistance (or impedance) measured in the pulse. In some examples, the termination criterion is a temporal condition, such as, for example, a time duration, predetermined or calculated based on some condition.

Electrical impedance is complex and, as such, includes a real component (resistance) and an imaginary component (reactance). This document describes techniques using impedance or resistance. It should be understood that where complex impedance values are available, such values can be used in place of resistance values. Conversely, where no complex impedance values are available, resistance values can be used instead unless otherwise stated.

Components of electrosurgical devices, such as electrodes and other intended tissue contacting components, can be coated to provide various effects, such as heat retention, energy concentration, non-stick properties, hydrophobic properties, wear resistance, low friction, etc.

In addition, many of the techniques below describe delivering electrosurgical energy to biological tissue. Unless indicated to the contrary, each of these techniques can deliver the electrosurgical energy using either power-controlled or voltage-controlled techniques. In a power-controlled implementation, a control circuit can control delivery of electrosurgical energy using a product of the voltage applied across the engaged biological tissue and the electrical current, e.g., according to a plan or schedule. For example, the control circuit can control delivery of a constant power or a monotonically increasing power during a particular phase, e.g., drying phase.

This document describes, among other things, one or more techniques for providing electrotherapy, which can be provided according to a treatment or other plan. The plan can include a recipe, prescription, regimen, methodology, or the like. In one example, the plan includes a waveform of energy application to tissue. Examples of a waveform may include different phases of energy application, each of which may include a delta of energy application. In one example, the energy application includes radio frequency (RF) energy application, although the invention is not so limited.

The plan can include one or more temporal aspects, such as a schedule, such as can include occurrence or recurrence (or inhibition or suppression) timing, frequency, type, relative combination (e.g., coagulation relative to cutting) or the like. The plan can include electrotherapy waveform information, such as can include pulse width, duty cycle, on duration, off duration, repetition rate, amplitude, phase, or the like. The plan need not be static or a priori in nature, but can include one or more dynamic aspects, such as can be modified or governed, such as by diagnostic, operational, or other information obtained during or between electrotherapy delivery instances, including in a closed-loop, or other feedback manner. One or more aspects of the plan can be tailored, such as to the specific patient, to a sub-population of patients such as who share one or more specified characteristics, or a population of patients, such as can be based on stored patient data, or by user input such as which may be provided by the patient or by a caregiver. The plan can include one or more conditional aspects, such as can include one or more branch conditions, such as can be determined using a patient characteristic, a diagnostic measure, an efficacy determination, or an operational characteristic of the device or its environment. Such branch conditions may be determined automatically, by the device, e.g., without requiring user input, or may involve user input, such as can be provided before, during, or after one or more portions of operations of the electrotherapy device according to the plan. The plan can involve communicating with or using another device, such as to receive or provide one or any combination of inputs, outputs, or instructions, operating parameters, or measured data. One or more aspects of the plan can be recorded or encoded onto a medium, such as a computer or other machine-readable medium, such as can be a tangible medium.

In one example, the plan anticipates, and includes an amount of heating that is applied to the tissue as a result of residual heat in an electrode contained by a thermally insulative coating that covers, or partially covers the electrode. Recognizing that this heat is available, and utilizing a selected coating material, coating thickness, and other coating variables to control a rate of heat transfer facilitates an additional component of a plan as described above. In one example a control circuit, as described in examples below, is programmed to indicate a hold time where a coated electrode remains in contact with the tissue although active energization of the electrode has ceased. During this hold time, residual heat from the electrode is applied to the tissue in a controlled rate and manner that is determined by the coating. A plan that takes this residual heat into account can provide additional tissue modification with a unique profile as determined by the coating, and the additional tissue modification is achieved without any additional energy applied to the electrode.

In a voltage-controlled implementation, the control circuit can control the voltage of the electrosurgical energy delivered, e.g., according to a plan, regimen, or schedule. For example, the control circuit can control delivery of a constant voltage or a monotonically increasing voltage during a particular phase, e.g., drying phase.

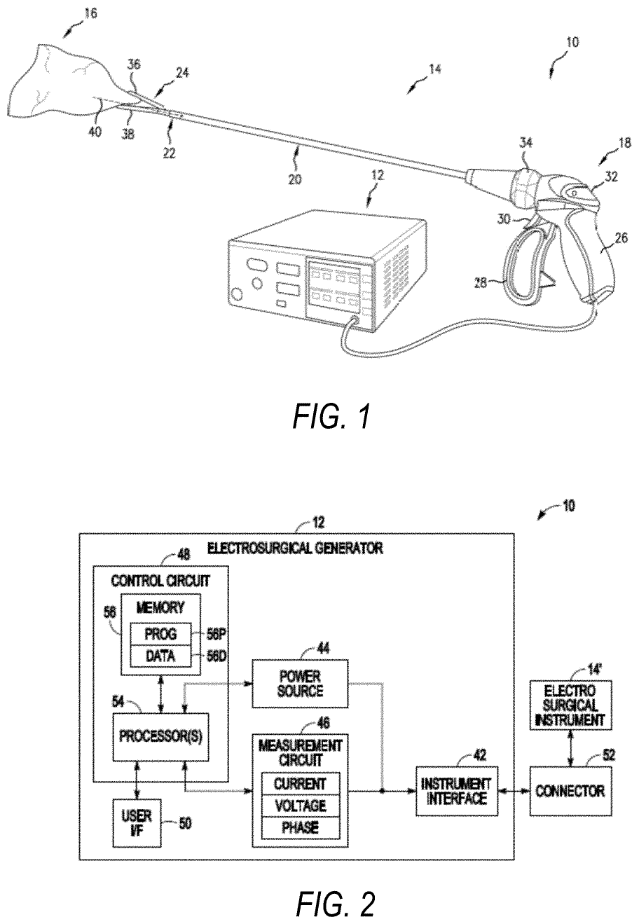

FIG. 1 is a perspective view of an electrosurgical system providing electrotherapy to biological tissue of a surgical patient. In FIG. 1, electrosurgical system 10 includes electrosurgical generator 12 and forceps 14, which is shown engaging biological tissue 16. Electrosurgical generator 12 is generating an electrotherapeutic signal which is provided to engaged biological tissue 16 via forceps 14. Although FIG. 1 depicts forceps 14 engaging and delivering the electrotherapeutic signal to biological tissue 14, various types of electrosurgical instruments, such as those disclosed above, can be used for such purposes.

Various types of forceps as well can be used for delivering the electrotherapeutic signal to biological tissue 14. For example, forceps 14 can be medical forceps, cutting forceps, or an electrosurgical forceps (e.g., monopolar or bipolar forceps). Forceps 14, in some examples, can be used for medically related procedures, such as open and/or laparoscopic medical procedures to manipulate, engage, grasp, cut, cauterize, seal, or otherwise affect a vessel, biological tissue, vein, artery, or other anatomical feature or object.

As illustrated in FIG. 1, forceps 14 includes hand piece 18, shaft assembly 20, knife blade assembly 22, and gripping assembly 24. In some examples, such as the illustrated example of FIG. 1, forceps 14 is electrically connected to electrosurgical generator 12, which generates the electrotherapeutic signal and provides the generated electrotherapeutic signal to forceps 14. Forceps 14 then electrically communicates the electrotherapeutic signal to gripping assembly 24 and/or to a remote pad, which can be employed for various electrosurgical techniques, such as cauterizing, sealing, or other such electrosurgical techniques.

Hand piece 18 includes handle 26, gripping lever 28, knife trigger 30, electrical therapy actuation button 32, and rotation wheel 34. Gripping assembly 24 includes first jaw member 36 and second jaw member 38. In one example, the first jaw member 36 may include a coating as described in more detail below. In one example, the second jaw member 38 may include a coating as described in more detail below. Shaft assembly 20 is connected at a proximal end to hand piece 18, and at a distal end to gripping assembly 24. Shaft assembly 20 extends distally from hand piece 18 in longitudinal direction 40 to gripping assembly 24.

Shaft assembly 20 functions to permit a portion of forceps 14 (e.g., gripping assembly 24 and a distal portion of shaft assembly 20) to be inserted into a patient or other anatomy while a remaining portion of forceps 14 (e.g., hand piece 18 and a remaining proximal portion of shaft assembly 20) are outside of the patient or other anatomy. Though illustrated in FIG. 1 as substantially straight, in other examples, shaft assembly 20 can include one or more angles, bends, and/or arcs. Shaft assembly 20 can be a cylinder with a circular, elliptical, or other cross-sectional profile, or other elongated member that extends from hand piece 18 to gripping assembly 24. In some examples, the shaft can be bendable, steerable or otherwise deflectable.

In some examples, such as the example of FIG. 1, shaft assembly 20 can include an elongated hollow member (e.g., a tubular outer shaft) that encloses knife blade assembly 22 and mechanical linkage to couple knife blade assembly 22 with knife trigger 30. In general, shaft assembly can be any elongated member having stiffness sufficient to transfer forces along longitudinal direction 40. Shaft assembly 20 also can include conductive elements (e.g., wires, a conductive outer shaft and/or a conductive inner shaft, etc.) to provide electrical communication between hand piece 18 and gripping assembly 24, so as to communicate an electrotherapeutic signal thereby.

Gripping lever 28, knife trigger 30, electrical therapy actuation button 32, and rotation wheel 34 of hand piece 18, each are configured to cause various actuations, usually at the distal end, of shaft assembly 20. For example, actuation of gripping lever 28 is configured to control operation of gripping assembly 24 at the distal end of shaft assembly 20. Gripping lever 28 is a gripping actuator that is movable between an open configuration position (illustrated in FIG. 1) and a closed configuration position in which gripping lever 28 is moved proximally toward handle 26. Movement of gripping lever 28 proximally toward handle 26 to the closed configuration position causes gripping assembly 24 to transition from the open configuration to the closed configuration. Movement of gripping lever 28 distally (e.g., release of gripping lever 28 to the open configuration position causes gripping assembly 24 to transition from the closed configuration to the open configuration.

Such transitions between the open and closed configurations of gripping assembly 24 are realized by one or more of first and second jaw members 36 and 38 moving between an open configuration (illustrated in FIG. 1), in which first and second jaw members 36 and 38 are spaced apart, and a closed configuration, in which the gap between first and second jaw members 36 and 38 is reduced or eliminated. Various electrosurgical instruments engage biological tissue 16 in various manners. In some electrosurgical instruments, such as the one illustrated in FIG. 1, first and second jaw members 36 and 38 are opposable to one another. In the depicted example first and second jaw members 36 and 38 are configured to clamp biological tissue 16 therebetween in a manner that provides electrical communication between opposable jaw members 36 and 38 via clamped biological tissue 16. Other electrosurgical instruments can engage biological tissue in other manners.

Mechanical linkage within shaft assembly 20 can be configured to cause one or more of first and second jaw members 36 and 38 to move between the open configuration and the closed configuration in response to actuation of gripping lever 28. One example mechanism for causing movement of a gripping assembly between the open and closed configurations can be found in U.S. Patent Publication No. 2017/0196579, entitled “FORCEPS JAW MECHANISM” and filed on Jan. 10, 2017 to Batchelor et al., the contents of which are hereby incorporated by reference in their entirety.

Actuation of knife trigger 30 is configured to control operation of knife blade assembly 22 located at the distal end of shaft assembly 20. Knife blade assembly 22 is configured to cut, excise, or otherwise affect biological tissue or other object(s) clamped between first and second jaw members 36 and 38. Knife trigger 30 is a knife blade actuator that is movable between a retracted configuration position (illustrated in FIG. 1) and a deployed or extended configuration position in which knife trigger 30 is moved proximally toward handle 26 to cause knife blade assembly 22 to cut biological tissue 16, which is clamped between first and second jaw members 36 and 38. Movement of knife trigger 30 proximally toward handle 26 to the deployed configuration position causes a cutting blade of knife blade assembly 22 to engage biological tissue 16, thereby cutting biological tissue 16. Movement of knife trigger 30 distally (e.g., release of knife trigger 30) causes the knife blade to retract from clamped biological tissue 16. Mechanical linkage, for example, within shaft assembly 20 can be configured to cause the knife blade to engage and retract from engaged biological tissue 16.

Rotation wheel 34 is configured to control rotational configuration of one or more of knife blade assembly 22, and gripping assembly 24 at the distal end of shaft assembly 20 and/or control rotational configuration of shaft assembly 20. Movement (e.g., rotation) of rotation wheel 34 causes rotation of one or more of shaft assembly 20, knife blade assembly 22, and gripping assembly 24 about an axis extending in longitudinal direction 40. Such rotational control can facilitate alignment of gripping assembly and/or knife blade assembly with clamped biological tissue 16.

Therapy actuation button 32 is configured to control generation and/or delivery of the electrotherapeutic signal to engaged biological tissue 16. Actuation of therapy actuation button 32 causes an electrotherapeutic signal, drawn from e.g., electrosurgical generator 12, to be applied to one or more of first and second jaw member 36 and 38, a remote pad (not illustrated), or other portions of forceps 14 to cauterize, seal, or otherwise electrically affect a patient or other anatomy. One example of a hand piece utilizing a gripping lever, knife trigger, rotation wheel, and therapy actuation button can be found in U.S. Pat. No. 9,681,883, entitled “FORCEPS WITH A ROTATION ASSEMBLY” and issued on Jun. 20, 2017 to Windgassen et al., the contents of which are hereby incorporated by reference in their entirety.

The aspects described herein can be used with any other type of electrotherapy device having one or more electrode, including but not limited to, scissor-style electrotherapy devices. In an illustrative example, scissor-style devices can include an upper handle coupled to a lower jaw that pivots relative to a lower handle that is coupled to an upper jaw to compress tissue between the upper and lower jaw during application of electrotherapeutic energy via the one or more electrode. Such scissor devices may not include all of the components described in the illustrative examples herein. For example, such scissors may not include a shaft or a rotation wheel.

FIG. 2 is a block diagram of an electrosurgical system for sealing biological tissue engaged by an electrosurgical instrument. In FIG. 2, electrosurgical system 10 include electrosurgical generator 12 and electrosurgical instrument 14′. Electrosurgical instrument 14′ can be any electrosurgical instrument configured to engage and deliver an electrotherapeutic signal to biological tissue. Electrosurgical generator 12 is configured to generate the electrotherapeutic signal, such as a high frequency (AC) electrical signal, that electrosurgical instrument 14′ delivers to engaged biological tissue 16.

In some examples, electrosurgical instrument 14′ is a forceps having a handpiece coupled to opposable jaw members via a shaft assembly, such as forceps 14 depicted in FIG. 1. In other examples, electrosurgical instrument 14′ is a conductive spatula, a conductive pad, or other electrosurgical device. These various types of electrosurgical instruments have various ways of engaging biological tissues (e.g., clamping, touching, surrounding, penetrating, radiating, etc.)

In one example, an electrode that is configured to engage biological tissue includes a coating covering at least a portion of the electrode. Several types of coatings are within the scope of the invention. Example coatings include wear resistant coatings, hydrophobic coatings, low adhesion coatings, low friction coatings, etc. In one example, a coating includes a thermally insulative property. In one example, a coating includes an electrical resistive property. In one example, a coating possesses two or more of the above listed properties.

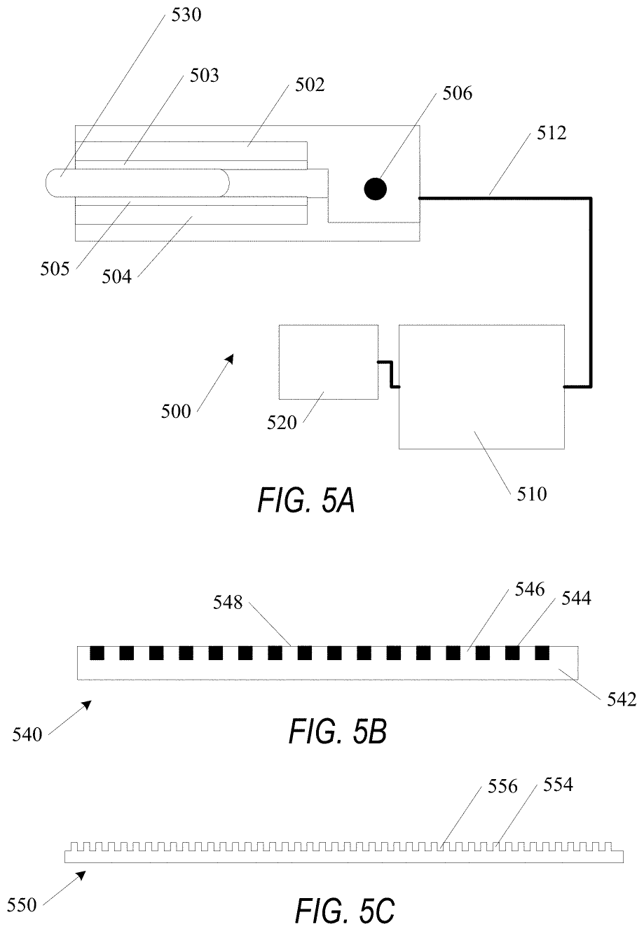

FIG. 5A shows selected portions of an electrosurgical device 500. A first electrode 502 and a second electrode 504 are shown. In the example of FIG. 5, the first electrode 502 and the second electrode 504 are jaws of an electrosurgical forceps, although the invention is not so limited. In the jaw example of FIG. 5A, a jaw pivot 506 is shown. Other example electrosurgical devices may only have a single electrode.

A first coating 503 is shown covering at least a portion of the first electrode 502. A second coating 505 is shown covering at least a portion of the second electrode 504. FIG. 5A illustrates the first electrode 502 and the second electrode 504 in contact with a portion of biological tissue 530.

A waveform generator 510 is shown coupled to the first electrode 502 and the second electrode 504 by circuitry 512. A controller 520 is coupled to the waveform generator 510. In one example, the controller 520 is configured to apply energy to one or more electrodes 502, 504 for a first duration to provide a first tissue modification, and indicate a second duration after the first duration to utilize heat in the electrode contained by a thermally insulative coating to provide a second tissue modification. In one example, the controller 520 is configured to apply energy to one or more electrodes 502, 504 wherein the electrode includes a resistive coating, and wherein the coating includes variations that define a number of first resistance regions and a number of second resistance regions interspersed with the number of first resistance regions, the second resistance regions having a lower resistance than the first resistance regions. This coating allows the controller to provide a higher concentration of the energy at the second resistance regions than at the first resistance regions to provide a tissue modification.

Any of a number of coating materials are within the scope of the invention. In one example selected coatings have properties that are determined by variables including material choice, thickness, etc. Variations in coating properties can be utilized to provide unique tissue modifications. One example includes electrical resistance variations. Example modifications to a coating that provide property variations include thickness variations, material variations, dopant concentration variations, etc. and geometric configurations of selected variations.

In one example, a coating property includes a selected thermal insulative property. Variables such as material choice, dopant selection, thickness selection, etc. provide a desired heat transfer property that is used by the controller to determine a hold time after application of energy to an electrode is turned off. By tailoring a heat transfer property of a coating, and combining the knowledge of the heat transfer property to programming of the controller, a secondary heat application rate and time can be controlled after the energy to the electrode is turned off.

In one example, a coating thickness is in a range between 5 and 350 nanometers, and perhaps preferably in a range between 15 and 300 nanometers. In a perhaps more preferred example, the thickness range may be between 95 and 300 nanometers. In a range below 95 nanometers, the coating may erode quicker than desired. In a range above 300 nanometers, a coating thickness may be undesirably insulative. This can depend on the coating type. In a perhaps more preferred example, the thickness may be at least 230 nanometers to provide at least one of: extending coating life further; to add an insulative effect (resistance/impedance) for measurement or monitoring by the electrosurgical generator; to control heat transfer characteristics. In a perhaps more preferred example, the thickness may be less than 300 nanometers to facilitate at least one of: control cost of the coating; to reduce time of drying the coating; to limit the insulative effect of the coating so as not to unduly inhibit the application of energy to a tissue.

Various ranges of coating thickness can be provided depending on the coating type and characteristics of the medical device they are applied to. Some examples are described. In one example, a coating thickness is greater than 220 nanometers. In one example, a coating thickness is in a range between 200 and 300 nanometers. In one example, a coating thickness is in a range between 220 and 300 nanometers. In one example, a coating thickness in a range between 230 and 300 nanometers. In one example, a coating thickness is in a range between 250 and 350 nanometers. In one example, a coating thickness is in a range between 90 and 200 nanometers. In one example, a coating includes a an electrical resistance of between approximately 5 and 15 ohms.

In one example, a coating includes an electrical resistance of between approximately 8 and 12 ohms. In one example, a coating includes a an electrical resistance of approximately 10 ohms. Coating material properties such as thickness may depend on bulk electrical and/or thermal properties of the material chosen. The above thickness examples apply to hexamethyldisiloxane (HDMSO) although the invention is not so limited. For other coating material choices, a thickness may be adjusted to provide similar desired electrical and thermal properties, and for the reasons described above.

Selections of coating that include these dimensions and resistivity can provide a number of advantages while maintaining other desired properties. For example, if a coating is too thick, energy application to an electrode may not be sufficiently close to a tissue to effectively modify the tissue. However, if a coating is too thin, it may not provide a desired thermally insulating property to provide residual heating over a desired secondary time after energy application to an electrode is turned off. Likewise, an electrical resistivity of a coating is important to maintain an effective modification to the tissue when energy is applied to the electrode.

Coating materials are also selected to provide coating properties such as hydrophobicity or wear resistance, or other properties as noted above. Selected coating materials may include polymer materials, monomer materials, ceramic materials, metal oxides or metal nitrides, glasses, metals or metal alloys, composite materials etc. Dopants can be used to modify coating properties in materials such as polymers. The dopants can be applied, either homogenously or in selected locations within the coating depending on the desired coating property. One example of a dopant material includes carbon, such as carbon black, graphite, graphene, carbon nanotubes, etc. Other examples of dopant materials include metal oxides, ceramics, etc. Dopants are chosen depending on a desired property. For example a carbon nanotube dopant may enhance electrical conductivity, while a ceramic dopant may enhance electrical resistivity.

FIG. 5B shows a coating 540 according to one example. The coating 540 may be used in examples in place of one or more of coatings 503, 505 from FIG. 5A. The coating 540 includes variations that define a number of first resistance regions 544 and a number of second resistance regions 546 interspersed with the number of first resistance regions 544. In the example of FIG. 5B, the number of first resistance regions 544 and the number of second resistance regions 546 are coplanar on an outer surface 548. In one example, a difference in resistivity between the first regions 544 and the second regions 546 is provided by different materials. and the number of second resistance regions 546 are coplanar on an outer surface 548. In one example, a difference in resistivity between the first regions 544 and the second regions 546 is provided by differences in a dopant between the regions 544 and 546.

In operation, it has been found that differences in resistivity provide a nucleation effect, where tissue adjacent to a coated electrode is more easily activated due to a lower energetic barrier due to low resistance regions. In effect, the presence of low resistance regions interspersed within higher resistance regions is more effective at treating tissue than a similar thickness coating without any variation in resistance across a surface.

In one example, the first regions 544 and second regions 546 are regularly interspersed, although the invention is not so limited. For example, a checkerboard type array of first regions 544 and second regions 546 may be included, or other arrangements that are not regular. In one example a regular pattern of several first regions 544 and second regions 546 provides a predictable response while still gaining the advantage of ease of activation of the tissue as described above.

FIG. 5C shows another example of a coating 550 according to one example. The coating 550 may be used in examples in place of one or more of coatings 503, 505 from FIG. 5A. The coating 550 includes thickness variations that define a number of first resistance regions 554 and a number of second resistance regions 556 interspersed with the number of first resistance regions 554. In the example of FIG. 5C, the variations in thickness provide the desired variations in resistivity in contrast with the local material variations described in FIG. 5B. In one example, both thickness variations and material variations may be used in conjunction with one another.

One advantage of material variations only, as illustrated in FIG. 5B, includes the ability to provide a smooth, coplanar surface 548 while still providing differences in resistivity. A smooth, coplanar surface 548 may reduce friction and/or may reduce an opportunity for fouling of cavities within the surface.

One advantage of asperities as first resistance regions 554 includes a potential added benefit of an ultrahydrophic surface, provided that dimensions of the first resistance regions 554 meet specific criteria for an ultrahydrophobic surface structure as described in more detail below.

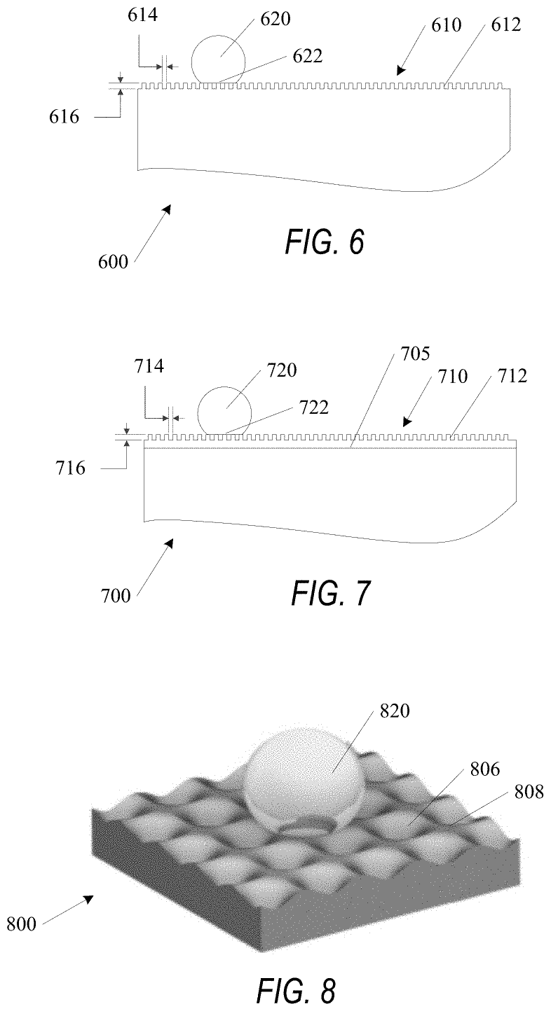

In one example, a coating may include a hydrophobic property. It may be advantageous to combine desired properties such as resistivity and hydrophobicity. One example of a hydrophobic coating may include a hydrophobic surface. One example of a hydrophobic surface that may vary and/or change over time includes an ultrahydrophobic surface structure. Examples of an ultrahydrophobic surface structure are described in FIGS. 6-8. FIG. 6 shows one example of a surface with a hydrophobic physical structure 610 on a substrate 602. As discussed in examples above, the hydrophobic physical structure 610 may be on all or a portion of a surface, and different hydrophobic physical structures 610 may be used on different surfaces or components of a cutting assembly. For example, the hydrophobic physical structure 610 may be on an entire outer surface of a cutting assembly. The hydrophobic physical structure 610 may be on only a portion of an outer surface of a cutting assembly.

As shown in FIG. 6, in one example, the hydrophobic physical structure 610 includes asperities 612 having a height 616 and a pitch 614. The hydrophobic physical structure 610 can be described by the following equation:

$${Λ}_{C} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 )}$$

where A is a contact line density, and Λ_{c} is a critical contact line density; ρ=density of the liquid droplet; g=acceleration due to gravity; V=volume of the liquid droplet; θₐ=advancing apparent contact angle; θ_{a,0}=advancing contact angle of a smooth substrate; γ=surface tension of the liquid; and w=tower wall angle.

The contact line density Λ is defined as a total perimeter of asperities over a given unit area.

In one example, if Λ>Λ_{c} then a droplet 620 of liquid are suspended in a Cassie-Baxter state. Otherwise, the droplet 620 will collapse into a Wenzel state. In one example when a Cassie-Baxter state is formed, an ultra-hydrophobic condition exists, and a low adhesion surface is formed. FIG. 6 illustrates a Cassie-Baxter state, where the droplet 620 rests on top of the asperities 612 at interface 622. Although rectangular asperities are shown for illustration purposes, the invention is not so limited. Asperity shapes are taken into account in the formula above, at least in the tower wall angle (w) term.

In the example of FIG. 6, the asperities are formed directly from a bulk material, and are not formed from a separate coating. One method of forming asperities directly from a bulk material includes chemical etching. Another example of forming asperities directly from a bulk material includes laser etching or ablation. Another example of forming asperities directly from a bulk material includes ion etching.

FIG. 7 shows another example of a surface with a hydrophobic physical structure 710 on a substrate 702. As discussed in examples above, the hydrophobic physical structure 710 may be on all or a portion of a surface, and different hydrophobic physical structures 710 may be used on different surfaces or components of a cutting assembly. For example, the hydrophobic physical structure 710 may be on an entire outer surface of a cutting assembly. The hydrophobic physical structure 710 may be on only a portion of an outer surface of a cutting assembly.

As shown in FIG. 7, in one example, the hydrophobic physical structure 710 includes asperities 712 having a height 716 and a pitch 714. However, in the example of FIG. 7, the hydrophobic physical structure 710 is formed as part of a coating 703 that forms a direct interface 705 with substrate 702. FIG. 7 illustrates a Cassie-Baxter state, where the droplet 720 rests on top of the asperities 712 at interface 722.

In one example, the asperities 712 are formed by application of nanoparticles to a surface of the substrate 702 to form the coating 703. In one example, the asperities 712 are formed by application of a continuous coating that assembles to form a nanoscale physical structure on a surface of the coating 703. In one example, the asperities 712 are formed by application of nanoparticles to a surface of the coating 703. In one example, the nanoparticles include a polymer. In one example, the nanoparticles include a monomer. In one example, the nanoparticles include a polysiloxane. In one example, the nanoparticles include hexamethyldisiloxane (HMDSO) particles. In one example, the nanoparticles include tetramethyldisiloxane (TMDSO) particles. In one example, the nanoparticles include fluorosilane particles. Other nanoparticle materials are also within the scope of the invention. In one example, a hydrophobic chemistry of the nanoparticle, in combination with a nano scale asperity structure as shown in FIG. 7 provide better hydrophobicity compared to a hydrophobic chemistry alone.

FIG. 8 shows one example of a laser etched surface 800 that includes hydrophobic physical structure as described above. In the example of FIG. 8, a gaussian hole array is formed by applying laser energy to a surface of a substrate 802 in a controlled regular pattern to form holes 806. A shape of the holes 806 is characterized as gaussian due to the energy distribution of laser energy in forming the array. In the example shown, a number of asperities 808 are formed in the process that may be spaced and arranged in an array that provides a Cassie-Baxter state as described above. A liquid droplet 820 is illustrated on the hydrophobic physical structure similar to the droplet 620 from FIG. 6, or the droplet 720 from FIG. 7.

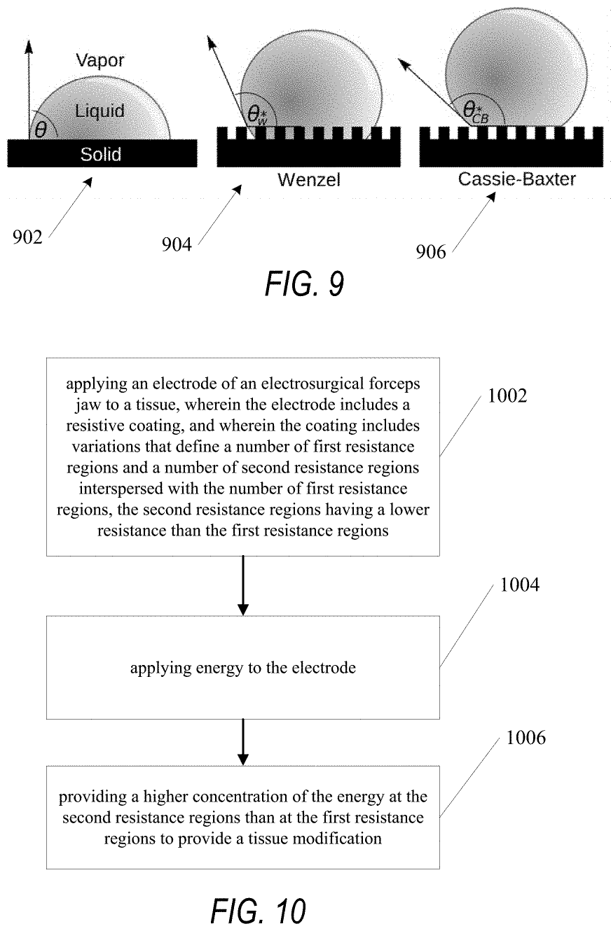

FIG. 9 shows three different states of hydrophobicity to further illustrate Cassie-Baxter hydrophobicity as discussed in examples above in comparison to other hydrophobic states. Illustration 902 shows a liquid droplet interacting with a smooth solid surface in air. The contact angle θ is shown at approximately 90° with respect to a plane of the solid surface. Illustration 904 shows a Wenzel state of a liquid droplet interacting with a number of asperities on a surface in air. In the Wenzel state, the contact angle θ_{W} is greater than 90° with respect to a plane of the solid surface. In the Wenzel state, the liquid droplet penetrates the asperities. Illustration 906 shows a Cassie-Baxter state of a liquid droplet interacting with a number of asperities on a surface in air. In the Cassie-Baxter state, the contact angle θ_{CB} is greater than 90° and greater than a Wenzel state contact angle (θ_{W}) for the same given liquid, solid and atmosphere (air) materials. In the Cassie-Baxter state, the liquid droplet does not fully penetrate the asperities.

Returning to FIG. 2, electrosurgical generator 12 includes instrument interface 42, electrical-energy source 44, measurement circuit 46, control circuit 48, and user interface 50. Instrument interface 42 can include signal drivers, buffers, amplifiers, ESD protection devices, and electrical connector 52, for example. Electrical connector 52 is configured to electrically couple electrosurgical instrument 14′ to electrosurgical generator 12 so as to provide electrical communication between electrosurgical generator 12 and electrosurgical instrument 14′. Such electrical communication can be used to transmit operating power and/or electrical signals therebetween. Electrosurgical instrument 14′, in turn, can provide electrical communication between electrical connector 52 and biological tissue engaged thereby.

Electrical-energy source 44 is configured to generate an electrotherapeutic signal to be delivered to the engaged biological tissue via electrically connected electrosurgical instrument 14′. One example of an electrotherapeutic signal includes an electrotherapeutic waveform. The generated electrotherapeutic signal can be controlled so as to obtain the desired result for a specific electrosurgical procedure. In one example, for example, the electrotherapeutic signal is configured to resistively heat the engaged biological tissue so as to surgically affect, such as seal, the engaged biological tissue. Such controlling of the electrotherapeutic signal will be further disclosed below.

Measurement circuit 46 is configured to measure one or more electrical parameters of biological tissue engaged by connected electrosurgical instrument 14′. Measurement circuit 46 is in electrical communication with connected electrosurgical instrument 14′ when electrosurgical generator 12 is electrically connected to electrosurgical instrument 14′ via electrical connector 52. Various examples of measurement circuit 46 are configured to measure various electrical parameters. For example, measurement circuit 46 can be configured to measure voltage difference delivered across and/or electrical current conducted by the engaged biological tissue. In some examples, measurement circuit 46 can be configured to measure phase angle between voltage difference delivered across and electrical current conducted by the engaged biological tissue. In some examples, measurement circuit 46 is configured to measure DC and or AC electrical parameters of the engaged biological tissue.

Measured parameters, such as voltage difference delivered across and/or electrical current conducted by the engaged biological tissue can be used to determine other electrical metrics. For example, measurements of voltage difference delivered across and/or electrical current conducted by the engaged biological tissue can be used to determine electrical resistance of the engaged biological tissue. Measurements of voltage difference delivered across and electrical current conducted by the engaged biological tissue, as well as the phase angle therebetween can be used to determine complex impedance of the engaged biological tissue. Measurements of voltage difference delivered across and electrical current conducted by the engaged biological tissue, as well as the phase angle therebetween also can be used to determine apparent power (VA) and/or real power (W) provide to the engaged biological tissue.

Such measurements of electrical parameters can be used for controlling an electrotherapeutic signal during delivery to an engaged biological tissue. For example, measurements of the voltage difference delivered across and measurements of the electrical current conducted by the engaged biological tissue can be used to determine and/or control the real power provided to the engaged tissue. This determined real power can then be compared with an electrotherapeutic schedule. Such a comparison could be used to generate an error signal. Measurements of electrical parameters can also be used to determine phase-control criteria for controlling phases of electrotherapy. Phase-control criteria can include criteria for commencement and termination of a phase, as well as criteria for intra-phase control.

Control circuit 48 is configured to take into account the presence and selected properties of coatings on electrodes. In one example, the control circuit 48 is configured to apply energy to one or more electrodes for a first duration to provide a first tissue modification, and indicate a second duration after the first duration to utilize heat in the electrode contained by a thermally insulative coating to provide a second tissue modification. In one example, the control circuit 48 is configured to apply energy to one or more electrodes wherein the electrode includes a resistive coating, and wherein the coating includes variations that define a number of first resistance regions and a number of second resistance regions interspersed with the number of first resistance regions, the second resistance regions having a lower resistance than the first resistance regions. This coating allows the controller to provide a higher concentration of the energy at the second resistance regions than at the first resistance regions to provide a tissue modification. Another example of a control circuit is shown as controller 520 in FIG. 5.

Control circuit 48 is electrically connected to electrical-energy source 44 and measurement circuit 46. Control circuit 48 causes electrical-energy source to provide an electrotherapeutic signal to biological tissue engaged by electrically connected electrosurgical instrument 14′. Control circuit 48 causes electrical-energy source 44 to generate the electrotherapeutic signal according to an electrotherapeutic schedule such that the generated electrotherapeutic signal is controlled for a specific electrosurgical procedure.

Various electrotherapeutic schedules can be used to effectuate various types of electrotherapy. For example, in some examples, real power (W) of the electrotherapeutic signal provided to the engaged biological tissue is controlled according to an electrical-power schedule. In other examples, voltage difference (V) of the electrotherapeutic signal delivered across the engaged biological tissue is controlled according to a voltage schedule. In other examples, electrical current (A) of the electrotherapeutic signal conducted by the engaged biological tissue is controlled according to an electrical-current schedule. In still other examples, apparent power (VA) of the electrotherapeutic signal provided to the engaged biological tissue can be controlled according to a voltage-amperage schedule.

Control circuit 48, for example, can cause electrical-energy source 44 to provide energy to engaged biological tissue, such that a product of a voltage difference across and an electrical current conducted by the engaged biological tissue is controlled according to the electrotherapeutic schedule. Control circuit 48 can use the comparison of the determined real power with an electrotherapeutic schedule to generate an error signal. Such an error signal can be used in a closed-loop feedback system that includes electrical-energy source 44, so as to generate the electrotherapeutic signal according to the electrotherapeutic schedule.

As illustrated in FIG. 2, control circuit 48 includes processor 54 and memory 56. Control circuit 48 can include a timer and/or a clock. In some examples, the timer and/or the clock are part of processor 54. In other examples, the timer and/or clock are separate from the processor 54. Processor 54, in one example, is configured to implement functionality and/or process instructions for execution within electrosurgical system 10. For instance, processor 54 can be capable of receiving from and/or processing instructions stored in program memory 56P. Processor 54 can then execute program instructions so as to cause electrical-energy source 44 to generate the electrotherapeutic signal according to a predetermined electrotherapeutic schedule. The predetermined electrotherapeutic schedule can be retrieved from data memory 56D, for example. Processor 54 can compare electrical parameters measured by measurement circuit 46 with the retrieved predetermined electrotherapeutic schedule. Processor 54 can send commands to electrical-energy source 44 and/or measurement circuit 46. Processor 54 also can also send or receive information from user interface 50.

In various examples, electrosurgical generator 12 can be realized using the elements illustrated in FIG. 2 or various other elements. For example, processor 54 can include any one or more of a microprocessor, a control circuit, a digital signal processor (DSP), an application specific integrated circuit (ASIC), a field-programmable gate array (FPGA), or other equivalent discrete or integrated logic circuitry.

Memory 56 can be configured to store information within electrosurgical system 10 during operation. Memory 56, in some examples, is described as computer-readable storage media. In some examples, a computer-readable storage media can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, memory 56 is a temporary memory, meaning that a primary purpose of memory 56 is not long-term storage. Memory 56, in some examples, is described as volatile memory, meaning that memory 56 does not maintain stored contents when power to electrosurgical system 10 is turned off. Examples of volatile memories can include random access memories (RAM), dynamic random access memories (DRAM), static random access memories (SRAM), and other forms of volatile memories. In some examples, memory 56 is used to store program instructions for execution by processor 54. Memory 56, in one example, is used by software or applications running on electrosurgical system 10 (e.g., a software program implementing electrical control of an electrotherapeutic signal provide to biological tissue engaged by an electrosurgical instrument) to temporarily store information during program execution, such as, for example, in data memory 56D.

In some examples, memory 56 can also include one or more computer-readable storage media. Memory 56 can be configured to store larger amounts of information than volatile memory. Memory 56 can further be configured for long-term storage of information. In some examples, memory 56 includes non-volatile storage elements. Examples of such non-volatile storage elements can include magnetic hard discs, optical discs, flash memories, or forms of electrically programmable memories (EPROM) or electrically erasable and programmable (EEPROM) memories.

User interface 50 can be used to communicate information between electrosurgical system 10 and a user (e.g., a surgeon or technician). User interface 50 can include a communications module. User interface 50 can include various user input and output devices. For example, User interface can include various displays, audible signal generators, as well switches, buttons, touch screens, mice, keyboards, etc.

User interface 50, in one example, utilizes the communications module to communicate with external devices via one or more networks, such as one or more wireless or wired networks or both. The communications module can include a network interface card, such as an Ethernet card, an optical transceiver, a radio frequency transceiver, or any other type of device that can send and receive information. Other examples of such network interfaces can include Bluetooth, 3G, 4G, and Wi-Fi radio computing devices as well as Universal Serial Bus (USB) devices.

FIG. 10 describes a method for modifying tissue. In operation 1002 an electrode of an electrosurgical forceps jaw is applied to a tissue. The electrode includes a resistive coating, and the coating includes variations that define a number of first resistance regions and a number of second resistance regions interspersed with the number of first resistance regions, the second resistance regions having a lower resistance than the first resistance regions. In operation 1004 energy is applied to the electrode, and in operation 1006 a higher concentration of the energy is provided at the second resistance regions than at the first resistance regions to provide a tissue modification.

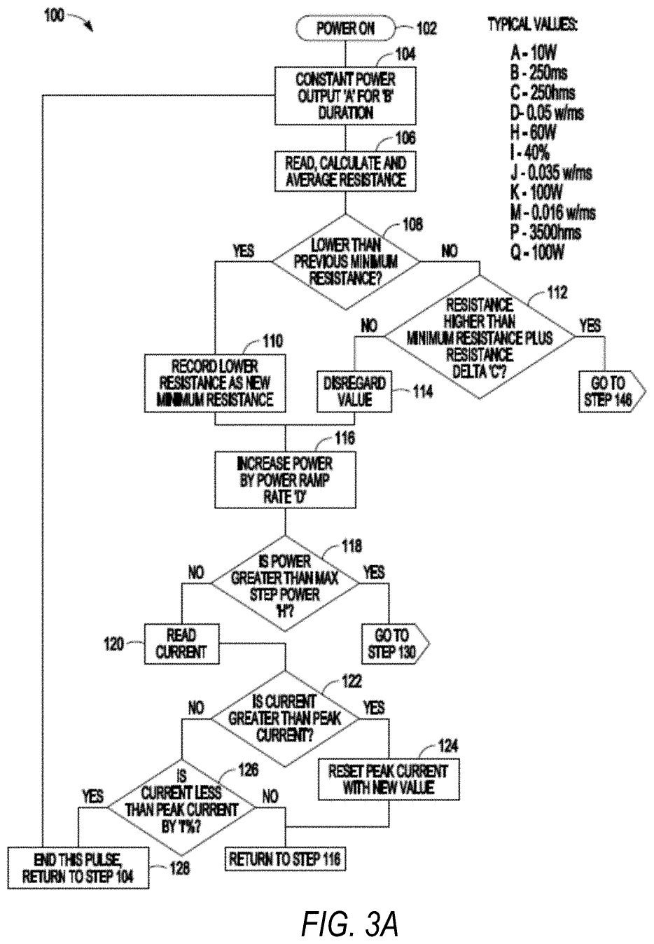

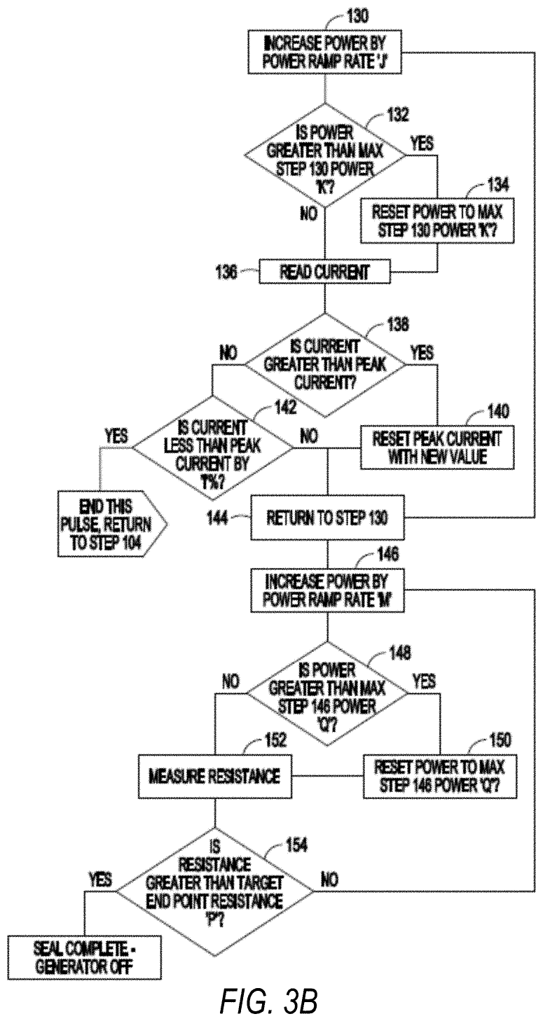

FIGS. 3A-3B are flow diagrams of a non-limiting example of a method for generating an electrotherapeutic signal for sealing a biological tissue engaged by an electrosurgical instrument. The flow diagrams utilize coated electrodes as described in examples above. Method 100 illustrated in FIGS. 3A-3B can be used with an electrosurgical system such as electrosurgical system 10 depicted in FIGS. 1-2. Using various techniques described below, an electrosurgical generator can control an energy delivery of the therapeutic signal provided to the biological tissue during a portion of a therapeutic phase according to an incremental change in energy delivery as a function of a change in a measured electrical parameter of the biological tissue. In some examples, a control circuit can control the electrical power of the therapeutic signal provided to the biological tissue during a portion of a therapeutic phase according a therapeutic plan, such as by controlling the power during the phase that provides the tissue modification.

For example, a control circuit can incrementally modify the power as a function of current. In some examples, the function of current is a function of a change in current. The change in current can be the change in the current over the course of a pulse and, as such, can look more like a current value. In some examples, the function of current is a function of an instantaneous measured change in current and, as such, can look more like the slope of the current function. The control circuit can modify the power based upon either of these—current or instantaneous change in current. In some examples, the function of the instantaneous measured change in current is a linear function. In other examples, the control circuit can incrementally modify the power as a function of resistance, such as when using a voltage-controlled technique.

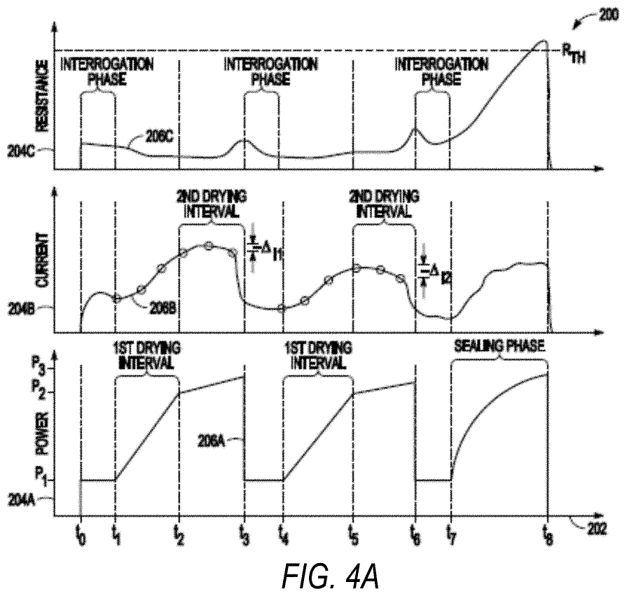

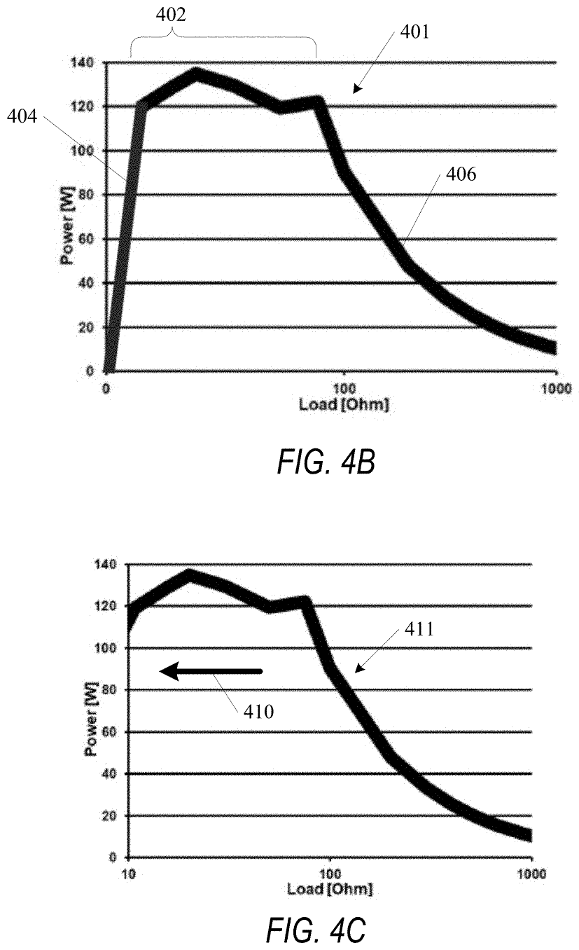

As seen in FIG. 4, in some examples, the system can control the electrical power of the therapeutic signal provided to the biological tissue during a portion of the therapeutic phase using a pre-defined power curve. In some examples, the pre-defined power curve can include two or more linear portions.

It should be noted that the FIGS. 3A and 3B and FIG. 4 are non-limiting specific examples used for purposes of explanation.

In some examples, the method can switch from using a power-controlled technique to using a voltage-controlled technique. In a voltage-controlled technique, current can be capped but allowed to move freely according to the responding impedance, which can allow for a variable power delivery. For example, the control circuit can deliver a pulse using power-controlled techniques and as the resistance increases, approaches a boiling condition, or reaches a threshold, the system can switch to a voltage-controlled technique. In this manner, at the beginning the system can take advantage of power-controlled techniques to deliver energy faster, but closer to boiling the system can switch over to voltage-controlled techniques, which can be more responsive. In some implementations that use a voltage-controlled technique, the system can control the electrical power of the therapeutic signal provided to the biological tissue during a portion of the therapeutic phase using a pre-defined voltage curve. In some examples, the pre-defined voltage curve can include two or more linear portions.

In FIG. 3A, method 100 begins at step 102, in which the electrosurgical system 10 (depicted in FIGS. 1-2) is powered on. Then, at step 104, an interrogation phase begins, in which control circuit 48 (depicted in FIG. 2) causes electrical-energy source 44 (depicted in FIG. 2) to provide an interrogation signal, such as an interrogation pulse, to the engaged biological tissue during the interrogation phase. Power (W) of the provided interrogation signal is controlled according to an interrogation schedule. In some examples, the power levels provided to the engaged biological tissue during the interrogation phase can be low so as to cause little or no tissue effect. Such low power levels can be provided for the purpose of obtaining measurements of electrical properties of the engaged biological tissue. Such measurements are sometimes obtained before electrotherapy is provided so as to obtain a pre-electrotherapy measurement. In some examples, the interrogation schedule is indicative of providing constant electrical power during the interrogation phase. Such a schedule can be called a constant power schedule. In some examples, control circuit 48 terminates the interrogation phase after a predetermined time duration.

At step 106, controller 48 causes measurement circuit 46 (depicted in FIG. 2) to measure a first electrical resistance of the engaged biological tissue during an interrogation phase. The first time that step 106 is performed this measured electrical resistance is a reference resistance. Then, at step 108, control circuit 48 compares the measured electrical resistance with a minimum resistance previously measured (if any). If, at step 108, the measured electrical resistance is lower than the minimum resistance, then the method advances to step 110, where the measured electrical resistance is recorded as the new minimum value, and then the method advances to step 116 (where a first interval of the drying or desiccation phase begins). If, however, at step 108, the measured electrical resistance is greater than the minimum resistance, then the method advances to step 112, where control circuit 48 compares the measured electrical resistance with a sum of the minimum resistance and a predetermined resistance delta. If, at step 112, the measured electrical resistance is less than the sum of the minimum resistance and a predetermined resistance delta, then the method advances to step 114, where the measured electrical resistance is disregarded. If, however, at step 112, the measured electrical resistance is greater than the sum of the minimum resistance and a predetermined resistance delta, then the method advances to step 146 illustrated in FIG. 3B. In one example, the minimum resistance is a baseline resistance as used in compensating for coating wear.

At step 116, a first interval of the drying or desiccation phase begins, such as where tissue modification occurs, in which control circuit 48 causes electrical-energy source 44 to provide a first drying signal, such as a first drying pulse, to the engaged biological tissue during the first drying interval of the drying phase. Power (W) of the provided first drying signal is controlled according to a first drying schedule or plan, such as using a pre-defined power curve, such as having a linear ramp rate. In some examples, the first drying schedule or plan is a monotonically-increasing power schedule, such as shown in the bottom graph in FIG. 4 between times t₁ and t₂.

Then, at step 118, control circuit 48 compares the provided power to a first threshold value, such as a first predetermined maximum power. If, at step 118, the provided power is greater than the first predetermined maximum power, then the method advances to step 130 illustrated in FIG. 3B, such as shown in the bottom graph in FIG. 4 between times t₂ and t₃, which depicts a second drying interval of the drying phase. In some examples that include a second drying interval, the control circuit 48 can reduce the ramp rate at block 130, such as shown in the bottom graph in FIG. 4 between times t₂ and t₃. In this manner, the control circuit 48 can modify the energy delivery during a first pulse, such as a first drying pulse, in response to the measured, e.g., intermittently, first electrical parameter of the engaged biological tissue meeting a first threshold value.

The system can measure, e.g., intermittently, the first electrical parameter, such as an electrical current and, in response to the measured electrical current of the engaged biological tissue satisfying a first threshold value, such as a predetermined value, reduce or terminate the energy delivery during the therapeutic phase. In some examples, the predetermined value is an absolute current threshold value. In some examples, the predetermined value is a threshold value that can change depending on a pulse count. In some examples, the predetermined value is a change in current relative to an initial current measurement. In some examples, the predetermined value is a change in current relative to a maximum current measurement during a pulse of the therapeutic signal.

If, however, at step 118, the provided power is less than the first predetermined maximum power, then the method advances to step 120, where control circuit 48 causes measurement circuit 46 to measure a first electrical parameter, such an impedance or an electrical current conducted by the engaged biological tissue.

At step 122, control circuit 48 compares the measured electrical current (or impedance), e.g., a first electrical parameter, for this pulse with the maximum electrical current previously measured (if any), e.g., a threshold value. If, at step 122, the measured electrical current is greater than the maximum electrical current, then the method advances to step 124, where the measured electrical current is recorded as the new maximum value, and then the method returns to step 116 so as to continue the first drying interval of the drying phase by modifying the energy delivery during the first pulse. If, however, at step 122, the measured electrical current is less than the maximum electrical current, then the method advances to step 126, where control circuit 48 compares the measured electrical current with a predetermined fraction of the maximum electrical current.

If, at step 126, the measured electrical current, e.g., a measured first electrical current, is greater than the predetermined current threshold, e.g., a measured second electrical current, then the method returns to step 116 so as to continue the first drying interval of the drying phase. In some examples, the predetermined current threshold can be a ratio or fraction of the maximum electrical current, such as 0.9, 0.8, 0.66, 0.5, and 0.4. for example. In other words, control circuit 48 can continue the drying signal or pulse in response to a ratio of the measured first electrical current to the measured second electrical current exceeding a predetermined factor indicating there has not been a phase change of liquid in the engaged biological tissue. In other examples, the predetermined current threshold can be a difference rather than a ratio.

If, however, at step 126, the measured electrical current is less than the predetermined fraction of the maximum electrical current, then the method advances to step 128, where the first drying pulse of the first drying interval of the drying phase is terminated. The method then returns to step 104 so as to repeat the interrogation phase, after which the drying phase can be repeated or a sealing phase can begin. In other words, the system can monitor electrical current during a therapeutic phase to determine when that therapeutic phase should end.

In some examples and in contrast to determining whether the measured electrical current is less than the predetermined faction of the maximum electrical current at step 126, the control circuit 48 can determine whether the measured electrical current is less than the predetermined fraction (or offset) of a current value measured at a predetermined time interval following the initiation of the pulse. For impedance monitoring systems, the control circuit 48 can determine whether the measured impedance is greater than the predetermined fraction (or offset) of a resistance value measured at a predetermined time interval following the initiation of the pulse.

At step 130 (depicted in FIG. 3B), a second interval of the drying phase begins, in which control circuit 48 causes electrical-energy source 44 to provide a second drying signal, such as a second drying pulse, to the engaged biological tissue during the second drying interval of the drying phase. It should be noted that although first and second drying intervals of a drying phase are shown in FIGS. 3A and 3B, there need not be a second drying interval of the drying phase. Rather, in some examples, the drying phase can terminate during the first drying interval. Power (W) of the provided second drying signal, such as a second drying pulse, is controlled according to a second drying schedule or plan, such as using a pre-defined power curve. In a power-controlled (or voltage-controlled or current-controlled) technique, the system can control the setting of the actuation energy level. Power (or voltage or current) constraint refers to a ceiling or threshold that the controlled current is not to cross, or there is an error state.

In other examples, Voltage (V) across the engaged biological tissue is controlled during the second drying interval. In a voltage-controlled technique, the system can control the setting of the actuation energy level. Voltage constraint refers to a ceiling or threshold that the controlled voltage is not to cross, or there is an error state. In voltage-controlled implementations, the control circuit can monitor the voltage of the therapeutic signal and when the threshold or ceiling is met, the control circuit can maintain the voltage at the threshold. In some voltage-controlled implementations, the voltage can be capped at a ceiling. In other voltage-controlled implementations, the voltage can be time variant.

In the depicted example, the second drying interval uses a second drying schedule or plan that is a monotonically-increasing power schedule. In some examples, for example, the second drying schedule or plan is a linearly-increasing power schedule. Then, at step 132, control circuit 48 compares the provided power to a second predetermined maximum power. If, at step 132, the provided power is greater than the second predetermined maximum power, then the method advances to step 134, where control circuit 48 causes electrical-energy source 44 to provide power equal to the second predetermined maximum power, e.g., a power ceiling, and then method 100 advances to step 136. If, however, at step 132, the provided power is less than the second predetermined maximum power, then the method advances to step 136, where control circuit 48 causes measurement circuit 46 to measure electrical current conducted by the engaged biological tissue.

At step 138, control circuit 48 compares the measured electrical current with maximum current previously measured. If, at step 138, the measured electrical current is greater than the maximum electrical current, then the method advances to step 140, where the measured electrical current is recorded as the new maximum value, and then the method returns to step 130 so as to continue the second drying phase. If, however, at step 138, the measured electrical current is less than the maximum electrical current, then the method advances to step 142, where control circuit 48 compares the measured electrical current with a predetermined fraction of the maximum electrical current. If, at step 142, the measured electrical current is greater than the predetermined ratio or fraction of the maximum electrical current, then the method returns to step 130 so as to continue the second drying interval of the drying phase. In other words, control circuit 48 can reduce the drying signal or pulse in response to a ratio of the measured first electrical current to the measured second electrical current exceeding a predetermined factor indicating a phase change of liquid in the engaged biological tissue. In other examples, the predetermined current threshold can be a difference. If, however, at step 142, the measured electrical current is less than the predetermined faction of the maximum electrical current, then the method can exit the second interval of the drying phase and return to step 104 so as to repeat the interrogation phase, after which the drying phase can be repeated or a sealing phase can begin. In other words, the system can monitor electrical current during a therapeutic phase to determine when that therapeutic phase should end.

At step 146, a sealing or coagulation phase begins in which control circuit 48 causes electrical-energy source 44 to provide a sealing signal, such as a sealing pulse, e.g., a second pulse, to the engaged biological tissue during the sealing phase, such as shown in the bottom graph in FIG. 4 between times t₇ and t₈. Power (W) of the provided sealing signal, such as a sealing pulse, is controlled according to a sealing schedule or plan. In some examples, the sealing schedule or plan is a monotonically-increasing power schedule. Then, at step 148, control circuit 48 compares the provided power to a third predetermined maximum power. It should be note that this is an example of a predetermined power curve, which happens to have a constant power domain. If, at step 148, the provided power is greater than the third predetermined maximum power, then the method advances to step 150, where control circuit 48 causes electrical-energy source 44 to provide power equal to the third predetermined maximum power, and then method 100 advances to step 152 to measure, e.g., intermittently, a second parameter of the engaged biological tissue, such as the resistance of the tissue. If, however, at step 148, the provided power is less than the third predetermined maximum power, then the method advances to step 152, where control circuit 48 causes measurement circuit 46 to measure electrical resistance of the engaged biological tissue.

At step 154, control circuit 48 compares the measured electrical resistance with a second threshold value, such as a calculated termination resistance value. In some examples, the calculated termination resistance value resistance is calculated based on the reference resistance measured at step 106, e.g., the first resistance. For example, the termination resistance value can be a predetermined factor times the measured reference resistance. In some examples, the termination resistance value can be a sum of a predetermined resistance delta and either the measured reference resistance or a minimum value of the resistance measured during that phase or a previous phase. In some examples, the target resistance is the predetermined delta resistance, where the predetermined delta resistance is a change in resistance relative to a minimum resistance measurement during a pulse of the therapeutic signal.

If, at step 154, the measured electrical resistance is less than the calculated termination resistance, then the method returns to step 146 so as to continue the sealing phase. If, however, at step 154, the measured electrical resistance is greater than the calculated termination resistance, then the sealing phase is terminated, and the method ends. In other words, in response to the measured, e.g., intermittently, impedance meeting a second threshold value, such as changing by a predetermined delta impedance value, for example, the method can modify the energy delivery of the second pulse, such as by reducing or terminating the energy delivery during this therapeutic phase, such as a sealing phase.

In some non-limiting examples, the method shown in FIGS. 3A and 3B can be implemented by a system such that the control circuit can monitor a first electrical parameter, such as an electrical current, in a first therapeutic phase, such as a drying phase, and reduce or terminate a first pulse based on the first electrical parameter, and monitor a second electrical parameter, such as an impedance, in a second therapeutic phase, such as a sealing phase, and reduce or terminate a second pulse based on the second electrical parameter.

FIG. 4A is a graph depicting non-limiting examples of an electrotherapeutic schedule or plan used to control electrical power provided to biological tissue being sealed. In FIG. 4A, Graph 200 has horizontal axis 202, vertical axes 204A-204C, and functional relations 206A-206C. Horizontal axis 202 is indicative of time (seconds). Horizontal axis has times t₀-t₈, which signify transition times between the interrogation, drying, and sealing phases disclosed in the discussion pertaining to method 100 for generating an electrotherapeutic signal for treating a biological tissue engaged by an electrosurgical instrument. These phases—the interrogation, first drying, and sealing phases—are also notated at various locations of graph 200. It should be noted that the graph of FIG. 4A is meant for purposes of explanation only. The graph of FIG. 4A depicts an example of a response and different tissues can react differently.

Vertical axis 204A is indicative of electrical power (W) provided to biological tissue engaged by an electrosurgical instrument. Functional relation 206A indicates a non-limiting example of a power/time relation corresponding to the electrotherapeutic signal generated based on the non-limiting example of a method 100 illustrated in FIGS. 3A-3B. Vertical axis 204B is indicative of electrical current conducted by the engaged biological tissue. Functional relation 206B indicates the electrical current/time relation pertaining to the electrical current conducted by the engaged biological tissue to which the electrotherapeutic signal generated via method 100 is provided. Vertical axis 204C is indicative of electrical resistance of the engaged biological tissue. Functional relation 206C indicates electrical-resistance/time relation corresponding to the electrical resistance of the engaged biological tissue to which the electrotherapeutic signal generated via method 100 is provided.

In some examples, the functional relation 206A can be a pre-defined power curve, including an interrogation phase, a drying phase, and a sealing phase. In the specific non-limiting example shown in FIG. 4, the drying phase depicts first and second drying intervals. From times t₀ to t₁, power/time relation 206A indicates the interrogation phase. In some examples, the duration of the interrogation phase is as short as is needed to obtain a reference measurement of the engaged biological tissue. For example, the duration of the interrogation phase can be less than 1.0, 0.5, 0.25, or 0.1 seconds. As indicated in graph 200, the interrogation phase is a constant-power schedule or plan having power P1 (W). From times t₀ to t₁, electrical current/time relation 206B indicates an interrogation rapid electrical current rise, followed by an electrical current plateau, which is then followed by a slight decrease in electrical current conducted by the engaged biological tissue. Because power is controlled to be constant throughout this interrogation phase, the voltage applied across the engaged biological tissue is inversely related (in a multiplicative sense as opposed to an additive sense) to the electrical current/time relation. The resistance of the engaged biological tissue can initially decrease as the temperature of the fluid in the tissue increases. Because this is the first time the interrogation phase is performed, the measured electrical resistance is not less than a minimum resistance previously measured, and therefore the method advances to the first drying phase.

From times t₁ to t₂, power/time relation 206A indicates the first interval of the drying phase. As indicated in graph 200, the first drying interval of the drying phase is an electrical-power schedule or plan that monotonically increases from powers P1 to P2 (W). From times t₁ to t₂, electrical current/time relation 206B indicates electrical current conducted by the engaged biological tissue increases throughout the first interval of the drying phase. Because power is controlled throughout this first interval of the drying phase according to a drying schedule or plan, a product of the voltage applied across the engaged biological tissue and the electrical current/time relation should yield power/time relation 206A. Although not depicted, in some examples, the electrical-resistance/time relation 206C can indicate that electrical resistance of the engaged biological tissue initially can decrease as the tissue warms, but then can increase as the tissue begins to dry during the first interval of the drying phase. Such increasing electrical resistance can indicate drying of the engaged biological tissue. Because the electrical current does not decrease below a fraction of a previously measured maximum electrical current before power/time relation 206A ramps to a predetermined threshold, the method advances to the second interval of the drying phase. If the current were to have dropped below the fraction of the previously measured maximum electrical current during this first interval of the drying phase, the subsequent second interval of the drying phase would not be necessary (e.g., it could be bypassed).

From times t₂ to t₃, power/time relation 206A indicates the second interval of the drying phase. As indicated in graph 200, the second interval of the drying phase is an electrical-power schedule or plan that monotonically increases from powers P2 to P3 (W). Using the techniques described above with respect to FIGS. 3A and 3B, a control circuit, such as the control circuit 48 of FIG. 2, can control an energy delivery of the therapeutic signal provided to the biological tissue during a portion of a therapeutic phase according to an incremental change in energy delivery as a function of a change in a measured electrical parameter of the biological tissue. For example, a control circuit can incrementally modify the power as a function of current. In some examples, the function of current is a function of an instantaneous measured change in current. In some examples, the function of the instantaneous measured change in current is a linear function. In other examples, the control circuit can incrementally modify the power as a function of resistance.

From times t₂ to t₃, electrical current/time relation 206B indicates electrical current conducted by the engaged biological tissue increases at the beginning of the second interval of the drying phase, but peaks and then decreases at the end of the second drying phase. It should be noted that a second interval of the drying phase may not be needed. In some examples, power can be controlled throughout this second interval of the drying phase such that a product of the voltage applied across the engaged biological tissue and the electrical current/time relation can yield a particular power/time relation 206A.

In some examples, the second interval of the drying phase is monotonically increasing, but at a slower rate of increase than that of the first interval of the drying phase. In other examples, the second interval of the drying phase is linearly increasing until the provided power equals a predetermined maximum level, after which time the provided power is held constant. Because the decrease in electrical current ΔI1, e.g., a measured change in current (such as at block 126 in FIG. 3A), results in a current that is less than a predetermined fraction of the maximum electrical current measured, the method returns to the interrogation phase, which is shown at time t₃. In other words, the change in electrical current ΔI1 causes the method to enter the interrogation phase at time t₃. It should be noted that in the non-limiting example shown in FIG. 4, the change in electrical current ΔI1 that causes the method to enter the interrogation phase is after time t₂. However, in other examples, the change in electrical current ΔI1 that causes the method to enter the interrogation phase can be after time t₁ during the first interval of the drying phase, and a second interval of the drying phase may not be needed. If, however, the decrease in electrical current ΔI1 had instead been less than the predetermined fraction of the maximum electrical current measured, then the method would have remained in the drying phase. As seen in FIG. 4, in some examples, the pre-defined power curve 206A can include two or more linear portions, such as shown between t₁ and t₂ and between t₂ and t₃.

From times t₃ to t₄, power/time relation 206A depicts the interrogation phase again. As indicated in graph 200, the interrogation phase is a constant-power schedule of power P1 (W). Because power is controlled to be constant throughout this interrogation phase, the voltage applied across the engaged biological tissue is inversely related (in a multiplicative sense as opposed to an additive sense) to the electrical current/time relation. Electrical-resistance/time relation 206C indicates that the electrical resistance of the engaged biological tissue is decreasing throughout this performance of the interrogation phase. Decreasing electrical resistance can be a result of condensing of fluid in the tissue or migration of fluid into the tissue. Because the measured electrical resistance is not greater than a sum of the reference resistance and a predetermined delta resistance, the method advances again to the first drying phase.

From times t₄ to t₅, power/time relation 206A indicates another first interval of the drying phase. The power/time relation from times t₄ to t₅ is similar to the power/time relation 206A from times t₁ to t₂ and, for purposes of conciseness will not be described in detail again.

From times t₅ to t₆, power/time relation 206A indicates another second interval of the drying phase. The power/time relation from times t₅ to t₆ is similar to the power/time relation 206A from times t₂ to t₃ and, for purposes of conciseness will not be described in detail again. Because power is controlled to be constant throughout this second interval of the drying phase, a product of the voltage applied across the engaged biological tissue and the electrical current/time relation should yield power/time relation 206A. Because the decrease in electrical current 412, e.g., a measured change in current (such as at block 142 in FIG. 3B), is less than a predetermined fraction of the maximum electrical current measured, the method returns to the interrogation phase.

From times t₆ to t₇, power/time relation 206A indicates another interrogation phase. The power/time relation from times t₆ to t₇ is similar to the power/time relation 206A from times t₃ to t₄ and, for purposes of conciseness will not be described in detail again. Because the measured electrical resistance is now greater than a sum of the reference resistance and a predetermined delta resistance, the method advances to the sealing phase.

From times t₇ to t₈, power/time relation 206A indicates the sealing phase. As indicated in graph 200, the sealing phase is an electrical-power schedule or plan that monotonically increases from power P1 to power P3 (W). From times t₇ to t₈, electrical current/time relation 206B indicates an increasing electrical current conducted by the engaged biological tissue throughout the sealing phase. Electrical-resistance/time relation 206C indicates that the electrical resistance of the engaged biological tissue is increasing this performance of the sealing phase. Increasing electrical resistance can be a result of drying and thereby sealing of the engaged biological tissue. Because the measured electrical resistance is now greater than a predetermined termination resistance, the sealing phase is terminated, and the method ends.

FIG. 4B shows a graph of power versus load for an uncoated electrode similar to electrodes described above. The power curve 401 includes an ideal operating power range 402, a low tissue resistance range 404 and a high tissue resistance range 406. In one example, a load as indicated in the Figure (total resistance) is equal to a sum of a resistance in the electrode, a resistance in the electronics of the device, and a resistance of the tissue being modified. In operation, a controller such as controller 520 or control circuit 48 applies a current to an electrode while in contact with tissue to be modified. Pursuant to Ohm's law, the power delivered from the application of the current is equal to the square of the current multiplied by the resistance in the circuit: P=I²R).

In the low tissue resistance range 404, because the tissue resistance portion of the load is low, the current required to reach the ideal operating power range 402 becomes quite high. An addition of an electrically resistive coating, when adjusted for by the controller, causes a shift of the power curve 401 along arrow 410 to power curve 411 illustrated in FIG. 4C. In one example, an electrically resistive coating includes a resistance of about 10 ohms, although the invention is not so limited. In such an example, the power curve 401 is shifted along arrow 410 to power curve 411 with a load along the x-axis beginning at 10 ohms as a result of the electrically resistive coating, as shown in FIG. 4C. If an electrically resistive coating includes another resistance, then the x-axis of FIG. 4C will have a different value for the shift 410.

As illustrated in FIG. 4C, a controller such as controller 520 or control circuit 48 is configured to adjust an applied current to apply an amount of power to the electrode such that when in operation, a resistance of the electrically resistive coating is accounted for, and the controller recognizes a range of acceptable tissue resistances shifted lower (to the left in FIG. 4C) than for an uncoated electrode of similar dimensions.

An amount of shift 410 between an uncoated electrode in FIG. 4B and a coated electrode in FIG. 4C is measurable. In one example, a controller such as controller 520 or control circuit 48 is configured to use the measurable shift to provide an adjusted energy delivery to tissue. An accurate measure compares an electrode of similar tissue contacting surface area in both FIG. 4B and FIG. 4C. In one example, an uncoated tissue contacting surface area of an electrode in FIG. 4B is within 2 percent of a coated tissue contacting surface area of an electrode in FIG. 4C. In one example, an uncoated tissue contacting surface area of an electrode in FIG. 4B is within 5 percent of a coated tissue contacting surface area of an electrode in FIG. 4C. In one example, an uncoated tissue contacting surface area of an electrode in FIG. 4B is within 10 percent of a coated tissue contacting surface area of an electrode in FIG. 4C.

To better illustrate the method and apparatuses disclosed herein, a non-limiting list of embodiments is provided here:

Example 1 includes an electrosurgical system. The system includes an electrosurgical device, including an electrode, a thermally insulative coating covering at least a portion of the electrode, a waveform generator configured to be coupled to the electrosurgical device, and a controller coupled to the waveform generator. The controller is configured to apply energy to the electrode for a first duration to provide a first tissue modification, and indicate a second duration after the first duration to utilize heat in the electrode contained by the thermally insulative coating to provide a secondary tissue modification.

Example 2 includes the electrosurgical system of example 1, wherein the thermally insulative coating includes a polymer coating.

Example 3 includes the example of any one of examples 1-2, wherein the thermally insulative coating includes hexamethyldisiloxane.

Example 4 includes the example of any one of examples 1-3, wherein the thermally insulative coating includes an electrical resistance modifying dopant.

Example 5 includes the example of any one of examples 1-4, wherein the electrical resistance modifying dopant includes carbon.

Example 6 includes the example of any one of examples 1-5, wherein the thermally insulative coating has a thickness in a range between 200 and 300 nanometers.

Example 7 includes the example of any one of examples 1-6, wherein the thermally insulative coating has a thickness in a range between 90 and 200 nanometers.

Example 8 includes the example of any one of examples 1-7, wherein the thermally insulative coating has an electrical resistance of approximately 10 ohms.

Example 9 includes the example of any one of examples 1-8, wherein the electrosurgical device includes an electrosurgical forceps.

Example 10 includes the example of any one of examples 1-9, wherein the thermally insulative coating includes an ultrahydrophobic surface structure.

Example 11 includes an electrosurgical device. The device includes an electrode, and a resistive coating covering at least a portion of the electrode, wherein the coating includes variations that define a number of first resistance regions and a number of second resistance regions interspersed with the number of first resistance regions, the second resistance regions having a lower resistance than the first resistance regions.

Example 12 includes the electrosurgical device of example 11, wherein the variations include differences in thickness within the coating.

Example 13 includes the electrosurgical device of any one of examples 11-12, wherein the resistive coating includes an electrical resistance modifying dopant.

Example 14 includes the electrosurgical device of any one of examples 11-13, wherein the electrical resistance modifying dopant includes carbon.

Example 15 includes the electrosurgical device of any one of examples 11-14, wherein the variations include different areal concentrations in electrical resistance modifying dopant concentrations within the coating.

Example 16 includes the electrosurgical device of any one of examples 11-15, wherein the resistive coating includes a polymer coating.

Example 17 includes the electrosurgical device of any one of examples 11-16, wherein the resistive coating includes hexamethyldisiloxane.

Example 18 includes the electrosurgical device of any one of examples 11-17, wherein the resistive coating has a thickness in a range between 200 and 300 nanometers.

Example 19 includes the electrosurgical device of any one of examples 11-18, wherein the resistive coating has a thickness in a range between 90 and 200 nanometers.

Example 20 includes the electrosurgical device of any one of examples 11-19, wherein the number of first resistance regions have an electrical resistance of approximately 10 ohms.

Example 21 includes the electrosurgical device of any one of examples 11-20, wherein the electrosurgical device includes an electrosurgical forceps.

Example 22 includes the electrosurgical device of any one of examples 11-21, wherein the resistive coating includes an ultrahydrophobic surface structure.

Example 23 includes a method. The method includes applying an electrode of an electrosurgical forceps jaw to a tissue, wherein the electrode includes a resistive coating, and wherein the coating includes variations that define a number of first resistance regions and a number of second resistance regions interspersed with the number of first resistance regions, the second resistance regions having a lower resistance than the first resistance regions, applying energy to the electrode, and providing a higher concentration of the energy at the second resistance regions than at the first resistance regions to provide a tissue modification.

Example 24 includes the method of example 23, wherein the number of first resistance regions have a thickness in a range between 200 and 300 nanometers.

Example 25 includes the method of any one of examples 23-24, wherein the number of second resistance regions are thinner than the number of first resistance regions.

Example 26 includes an electrosurgical system. The system includes an electrosurgical device, including an electrode, an electrically resistive coating covering at least a portion of the electrode, the electrically resistive coating having a thickness, a waveform generator configured to be coupled to the electrosurgical device, and a controller coupled to the waveform generator, the controller configured to adjust an applied current to apply an amount of power to the electrode such that when in operation, a resistance of the electrically resistive coating is accounted for, and the controller recognizes a range of acceptable tissue resistances shifted lower than for an uncoated electrode of similar dimensions.

Example 27 includes the electrosurgical system of example 26, wherein the electrically resistive coating includes hexamethyldisiloxane (HMDSO).

Example 28 includes the electrosurgical system of example of any one of examples 26-27, wherein the thickness is 220 to 300 nm.

Example 29 includes the electrosurgical system of example of any one of examples 26-28, wherein the thickness is 250 to 350 nm.

Example 30 includes the electrosurgical system of example of any one of examples 26-29, wherein the electrically resistive coating has a resistance between 5 ohms and 15 ohms.

Example 31 includes the electrosurgical system of example of any one of examples 26-30, wherein the electrically resistive coating has a resistance between 8 ohms and 12 ohms.

Example 32 includes the electrosurgical system of example of any one of examples 26-31, wherein the electrically resistive coating has a resistance of approximately 10 ohms.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the inventive subject matter has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The foregoing description, for the purpose of explanation, has been described with reference to specific example embodiments. However, the illustrative discussions above are not intended to be exhaustive or to limit the possible example embodiments to the precise forms disclosed. Many modifications and variations are possible in view of the above teachings. The example embodiments were chosen and described in order to best explain the principles involved and their practical applications, to thereby enable others skilled in the art to best utilize the various example embodiments with various modifications as are suited to the particular use contemplated.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

The terminology used in the description of the example embodiments herein is for the purpose of describing particular example embodiments only and is not intended to be limiting. As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

1. An electrosurgical system, comprising:
an electrosurgical device, including;
an electrode;
a thermally insulative coating covering at least a portion of the electrode;
a waveform generator configured to be coupled to the electrosurgical device; and
a controller coupled to the waveform generator, the controller configured to;
apply energy to the electrode for a first duration to provide a first tissue modification; and
indicate a second duration after the first duration to utilize heat in the electrode contained by the thermally insulative coating to provide a secondary tissue modification.

2. The electrosurgical system of claim 1, wherein the thermally insulative coating includes a polymer coating.

3. The electrosurgical system of claim 1, wherein the thermally insulative coating includes hexamethyldisiloxane.

4. The electrosurgical system of claim 1, wherein the thermally insulative coating includes an electrical resistance modifying dopant.

5. The electrosurgical system of claim 4, wherein the electrical resistance modifying dopant includes carbon.

6. The electrosurgical system of claim 1, wherein the thermally insulative coating has a thickness in a range between 200 and 300 nanometers.

7. The electrosurgical system of claim 1, wherein the thermally insulative coating has a thickness in a range between 90 and 200 nanometers.

8. The electrosurgical system of claim 1, wherein the thermally insulative coating has an electrical resistance of approximately 10 ohms.

9. The electrosurgical system of claim 1, wherein the electrosurgical device includes an electrosurgical forceps.

10. The electrosurgical system of claim 1, wherein the thermally insulative coating includes an ultrahydrophobic surface structure.

11. An electrosurgical device, comprising:
an electrode;
a resistive coating covering at least a portion of the electrode, wherein the coating includes variations that define;
a number of first resistance regions; and
a number of second resistance regions interspersed with the number of first resistance regions, the second resistance regions having a lower resistance than the first resistance regions.

12. The electrosurgical device of claim 11, wherein the variations include differences in thickness within the coating.

13. The electrosurgical device of claim 11, wherein the resistive coating includes an electrical resistance modifying dopant.

14. The electrosurgical device of claim 13, wherein the electrical resistance modifying dopant includes carbon.

15. The electrosurgical device of claim 13, wherein the variations include different areal concentrations in electrical resistance modifying dopant concentrations within the coating.

16. The electrosurgical device of claim 11, wherein the resistive coating includes a polymer coating.

17. The electrosurgical device of claim 11, wherein the resistive coating includes hexamethyldisiloxane.

18. The electrosurgical device of claim 11, wherein the resistive coating has a thickness in a range between 200 and 300 nanometers.

19. The electrosurgical device of claim 11, wherein the resistive coating has a thickness in a range between 90 and 200 nanometers.

20. The electrosurgical device of claim 11, wherein the number of first resistance regions have an electrical resistance of approximately 10 ohms.

21. The electrosurgical system of claim 11, wherein the electrosurgical device includes an electrosurgical forceps.

22. The electrosurgical system of claim 11, wherein the resistive coating includes an ultrahydrophobic surface structure.

23. A method, comprising:
applying an electrode of an electrosurgical forceps jaw to a tissue, wherein the electrode includes a resistive coating, and wherein the coating includes variations that define a number of first resistance regions and a number of second resistance regions interspersed with the number of first resistance regions, the second resistance regions having a lower resistance than the first resistance regions;
applying energy to the electrode; and
providing a higher concentration of the energy at the second resistance regions than at the first resistance regions to provide a tissue modification.

24. The method of claim 23, wherein the number of first resistance regions have a thickness in a range between 200 and 300 nanometers.

25. The method of claim 23, wherein the number of second resistance regions are thinner than the number of first resistance regions.

26. An electrosurgical system, comprising:
an electrosurgical device, including;
an electrode;
an electrically resistive coating covering at least a portion of the electrode, the electrically resistive coating having a thickness;
a waveform generator configured to be coupled to the electrosurgical device; and
a controller coupled to the waveform generator, the controller configured to adjust an applied current to apply an amount of power to the electrode such that when in operation, a resistance of the electrically resistive coating is accounted for, and the controller recognizes a range of acceptable tissue resistances shifted lower than for an uncoated electrode of similar dimensions.

27. The electrosurgical system of claim 26, wherein the electrically resistive coating includes hexamethyldisiloxane (HMDSO).

28. The electrosurgical system of claim 27, wherein the thickness is 220 to 300 nm.

29. The electrosurgical system of claim 27, wherein the thickness is 250 to 350 nm.

30. The electrosurgical system of claim 27, wherein the electrically resistive coating has a resistance between 5 ohms and 15 ohms.

31. The electrosurgical system of claim 27, wherein the electrically resistive coating has a resistance between 8 ohms and 12 ohms.

32. The electrosurgical system of claim 27, wherein the electrically resistive coating has a resistance of approximately 10 ohms.
